(19) United States
(12) Patent Application Publication
(10) Pub. No.: US 2006/0058402 A1
(43) Pub. Date: Mar. 16, 2006

(54) Sequestration of carbon dioxide

(76) Inventor: Donald James Highgate, Surrey (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/224,366

(22) Filed: Sep. 12, 2005

(30) Foreign Application Priority Data
Sep. 10, 2004 (GB) 0420116.6

(51) Int. Cl. C07C 27/26

(52) U.S. Cl. 518/726

(57) ABSTRACT

Hydrogen is used to manufacture hydrocarbons, utilising carbon extracted from the atmosphere or from an exhaust flow prior to release into the atmosphere.

### FIELD OF THE INVENTION

This invention relates to a process for the sequestration of carbon dioxide from the environment.

### BACKGROUND OF THE INVENTION

At present, the main focus of research on storage media is on geological sinks and the deep ocean. Geological storage includes deep saline formations (subterranean and sub-seabed), depleted oil and gas reservoirs, enhanced oil recovery, and unminable coal seams. Deep ocean storage includes direct injection of liquid carbon dioxide into the water column at intermediate depths (1000-3000 m), or at depths greater than 3000 m, where liquid CO₂ becomes heavier than sea water, so that it drops to the ocean bottom and forms a so-called “CO₂ lake”. The permanence of these methods is still to be established, but the intention is clear, i.e. to remove the material from the environment for a period of time long compared to a human lifetime, a definition which is intended hereafter when ‘permanent’ is used.

Alternatively, it is possible to take hydrogen and carbon dioxide obtained directly from a process plant or by extraction from the atmosphere, and combine them, to form hydrocarbon compounds. These hydrocarbons are not generally considered permanent (in the example of the production of alcohols and other hydrocarbons generally up to C₁₈) and are intended for use as fuels. This latter process is also incorrectly referred to as sequestration, but the result is entirely short term, i.e. until the fuel is reused.

### SUMMARY OF THE INVENTION

This invention is based on an appreciation of the utility of hydrogen combined with carbon dioxide to manufacture hydrocarbons. Preferably, the hydrogen used is “carbon-free” or “low carbon”, e.g. hydrogen derived by electrolysis using electricity generated by a “carbon-free” or “low carbon” process, wind power or solar power. The carbon dioxide may be extracted from the atmosphere, or from exhaust flows prior to release into the atmosphere.

### DETAILED DESCRIPTION OF THE INVENTION

The availability of carbon-free hydrogen, e.g. hydrogen derived by electrolysis using electricity generated by a carbon free process, wind power or solar power, combined with carbon dioxide extracted from the atmosphere, allows for:
(i) the generation of a “zero-carbon” secondary hydrocarbon fuel in the range C, to C₂₆.
The fuel thus produced is genuinely of zero environmental impact in regards to its additional effect upon the atmospheric carbon dioxide content, however the method can not properly be considered as a method of sequestration because the carbon dioxide is inevitably released back into the atmosphere during its subsequent use. However, the use of this zero-carbon secondary hydrocarbon fuel could lead to a reduction in primary fuel use, and is therefore considered beneficial.
(ii) the permanent sequestration of atmospheric carbon dioxide by the production of hydrocarbons in the range C₂₆ to C₇₀+
The hydrocarbons produced would be classified as permanent if they would not normally degrade to release significant amounts of CO₂ in normal atmospheric conditions. Ideally these products would have an economically viable application, e.g. as bitumen or pitch.

This latter process has the benefit of producing a product of industrial value that can be used (e.g. in building or road-making) without releasing the carbon dioxide back to the atmosphere; it therefore fulfils the requirement for a long-term permanent sequestration process. In addition, if the use of the product replaces concrete then the process has the potential to significantly reduce the national carbon dioxide burden.

Hydrocarbon synthesis has been employed by a number of different industries for a variety of purposes. Fischer-Tropsch (F-T) chemistry converts Syngas (a mixture of CO and H₂) into a mixture of mainly straight-chain hydrocarbons. The hydrocarbons include materials of varying carbon chain lengths and molecular weights. The use of the F-T process is well known for the production of alcohols. The F-T product distribution typically follows the single-parameter Anderson-Schulz-Flory (ASF) equation:
Wn=n(1−α)₂αn−1
where Wn is the weight fraction of product of carbon n, and α is the chain growth probability. The higher the value of α, the longer the average chain length of the hydrocarbons. In practice, there is often a deviation from the ideal ASF distribution; the extent of this deviation varies with the nature of the catalyst and the operating conditions.

Through suitable control of these parameters, the probability of producing higher length chain hydrocarbons can be increased, for example through the addition of certain transition metal oxides (e.g. ZrO₂) which act as an oxide promoter or the use of an iron or cobalt catalyst will promote an increased production of higher chain hydrocarbons with higher molecular weights.

A further improvement to the F-T process may be the use of irradiation either during or preceding the chemical process. Irradiation may lead to further increases in molecular weight of the hydrocarbons.

I claim:

1. A method for manufacturing a hydrocarbon wherein said method comprises combining hydrogen with carbon from the atmosphere or from an exhaust flow prior to release into the atmosphere.

2. The method according to claim 1, wherein the hydrogen is “carbon-free” or “low carbon” hydrogen.

3. The method according to claim 1, wherein the hydrocarbon is short-term and intended for use as a fuel.

4. The method according to claim 1, wherein the hydrocarbon is permanent.

5. The method according to claim 1, wherein the hydrogen is derived by photolysis.

6. The method according to claim 2, wherein the hydrogen is derived by electrolysis.

7. The method according to claim 6, wherein the electricity required for electrolysis is derived from a “carbon-free” or “low carbon” process.

8. The method according to claim 1, wherein the manufacture is via a Fischer-Tropsch reaction including a material that promotes the synthesis of higher chain hydrocarbons.

9. The method according to claim 1, wherein the method further comprises irradiation.
